(19) United States
(12) Patent Application Publication
(10) Pub. No.: US 2024/0238094 A1
(43) Pub. Date: Jul. 18, 2024

(54) HYDROGEL-COATED ORTHOPEDIC IMPLANTS

(71) Applicant: Duke University, Durham, NC (US)

(72) Inventors: Benjamin WILEY, Durham, NC (US); Huayu TONG, Durham, NC (US)

(21) Appl. No.: 18/559,337

(22) PCT Filed: May 4, 2022
(86) PCT No.: PCT/US2022/027593

§ 371 Date: Nov. 6, 2023

Related U.S. Application Data
(60) Provisional application No. 63/183,670, filed on May 4, 2021.

(51) Int. Cl. A61F 2/30; A61L 27/06; A61L 27/34; A61L 27/52

(52) U.S. Cl. CPC A61F 2/30756; A61L 27/06; A61L 27/34; A61L 27/52; A61F 2002/3092; A61F 2002/30971; A61L 2420/02

(57) ABSTRACT

Hydrogel-coated orthopedic implants with surfaces that mimic the mechanical and tribological properties of cartilage, and bases that enable integration with bone for long-term fixation, and methods of forming them.

### CROSS REFERENCE TO RELATED APPLICATIONS

This patent application claims priority to U.S. provisional patent application No. 63/183,670, titled “HYDROGEL-COATED ORTHOPEDIC IMPLANTS,” filed on May 4, 2021 and herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Every year, approximately 900,000 people in the United States suffer from damage to the articular cartilage that lines the ends of the bones, with the knee being most commonly affected. Articular cartilage lesions have a limited intrinsic ability to heal and often lead to osteoarthritis. Although treatment of cartilage lesions can alleviate debilitating pain and delay the need for a total knee replacement, current strategies for cartilage restoration including bone marrow stimulation (microfracture), autologous cartilage cell implantation, and osteochondral transplantation typically have high failure rates (e.g., 25-50% at 10 years), prolonged rehabilitation times (>12 months), can be very costly, and show decreasing efficacy in patients older than 40-50 years. Although alternatives such as focal joint resurfacing with traditional orthopedic materials (e.g. Cobalt-Chromium alloy, ultra-high-molecular-weight polyethylene) are being explored as an alternative strategy, these implants have limited ability to biologically integrate, and there are concerns they may contribute to joint degeneration through abnormal stress and wear on the opposing cartilage surface. It is widely acknowledged that a cost-effective procedure that can immediately restore the mechanical function of cartilage while enabling long-term biological integration is needed.

Hydrogels, polymer networks swollen with water, are a promising synthetic material for replacement of cartilage. However, there is currently no way to secure hydrogel into a cartilage defect site with the same shear strength as the junction between cartilage and bone.

### SUMMARY OF THE DISCLOSURE

This disclosure relates generally to artificial cartilage materials in implants suitable for repair of cartilage, including specifically methods and compositions for attaching polymer network hydrogel materials to a surface of an implant, as well as implants including polymer network hydrogels. In particular, described herein are methods and apparatuses for replacement of damaged cartilage with a synthetic hydrogel that allows securing of a hydrogel in a defect site with the same shear strength as the cartilage-bone interface. In some examples these methods and apparatuses may include bonding a hydrogel to a titanium base that can in turn bond to bone and may enable long-term fixation of the hydrogel. Unlike currently known methods of forming bonds to hydrogels, which do not have the shear strength of the cartilage-bone interface, the methods and apparatuses described herein may allow (for the first time) attaching of a hydrogel to a metal with approximately the same shear strength as the cartilage-bone interface.

Although adhesive cements may achieve shear strengths up to about 22 MPa between two porous titanium plugs, the same cements can typically only achieve a shear strength of about 3 MPa or less between a porous titanium and a hydrogel (such as a bacterial cellulose-reinforced hydrogel). As described in greater detail herein, the lower shear strength of the hydrogel on titanium may be due to delamination of the layers of cellulose nanofibers in the bacterial cellulose. The methods and apparatuses described herein may prevent or reduce delamination by reorienting the bacterial cellulose layers in the hydrogel so that they are perpendicular to the direction of shear in the implanted joint. This orientation of the bacterial cellulose may mimic the orientation of collagen nanofibers in the osteochondral junction. Reorientation of the bacterial cellulose may be achieved by wrapping the bacterial cellulose layers over the periphery of the metal plug and fixing them in place (e.g., with a clamp, such as but not limited to a shape memory alloy clamp), followed by infiltration of the hydrogel components into the bacterial cellulose.

Surprisingly, the average shear strength of a junction between a hydrogel (e.g., a 1-mm-thick hydrogel) and a metal as described herein exceeds the shear strength of a porcine cartilage-bone interface. The shear strength of attachment increases with the number of bacterial cellulose layers and with the addition of cement between the bacterial cellulose layers. This new method of attachment will be particularly useful in the creation of hydrogel-coated orthopedic implants for treatment of osteochondral defects but may be used in any method or apparatus in which it is helpful to couple a hydrogel to substrate (including, but not limited to a metal).

For example, described herein are implants, comprising: an implant body, the implant body having an engagement surface surrounded by an edge region that is substantially perpendicular to a perimeter of the engagement surface; one or more sheets of bacterial cellulose (BC) applied over the engagement surface and along the edge region; and a clamp securing the one or more sheets of BC material between the edge region and the clamp, wherein the one or more sheets of BC material over the engagement surface are infiltrated with a hydrogel material to form a BC-network hydrogel.

As used herein the term “substantially perpendicular” may refer to within about +/−15 degrees (e.g., +/−12.5 degrees, +/−10 degrees, +/−9 degrees, +/−8 degrees, +/−7 degrees, +/−6 degrees, +/−5 degrees, +/−4 degrees, +/−3 degrees, +/−2 degrees, +/−1 degree) of absolute perpendicular. For example, an edge region that is substantially perpendicular to a perimeter of the engagement surface may be arranged so that the edge region is approximately 90 degrees relative to the perimeter of the engagement surface (e.g., between 75 degrees and 105 degrees, between 77.5 degrees and 102.5 degrees, between 80 degrees and 100 degrees, between 81 degrees and 99 degrees, etc.).

The implant body may be formed of any appropriate material, including in particular biocompatible materials. For example, the implant body may be formed of titanium. The implant body may be porous; in some examples the engagement surface may be porous.

The engagement surface may be curved (e.g. convex, concave), or flat. The engagement surface and/or the edge region may include an adhesive between the one or more sheets of BC material and the engagement surface and/or edge region. In some examples just the bottom-most sheet of the one or more sheets of BC material is adhesively coupled to the implant. In some examples an adhesive is included between all or some of the multiple sheets of BC material. In any of these implants, the one or more sheets of bacterial cellulose comprises 3 or more sheets (e.g., 4 or more, 5 or more, 6 or more, 7 or more, etc.).

As used herein, a sheet of bacterial cellulose (BC) may refer to a substantially planar (e.g., flat) arrangement of bacterial cellulose that may be folded, bent, and/or laid onto another surface. Sheets of BC may be stacked atop each other and placed onto an implant surface. The sheet may be dry (e.g., freeze dried), or wet. A sheet of BC has a thickness, a length, and a width, in which the length and width are both typically much larger than the thickness (e.g., the length-to-thickness aspect ratio and the width-to-thickness aspect ratio of 5 or greater (e.g., 6 or more, 7 or more, 8 or more, 9 or more, 10 or more, etc.). In some examples a sheet of BC may be 1 mm or less.

The hydrogel material may be any appropriate material forming a BC-network hydrogel. In some examples the hydrogel material comprises polyvinyl alcohol (PVA). In some examples, the hydrogel material comprises PVA and poly(2-acrylamido-2-methyl-1-propanesulfonic acid (PAMPS). Alternatively, in some examples the hydrogel material comprises just PVA or PVA without PAMPS.

The implant body may comprise an elongated nail shape configured to be implanted into bone. For example, the implant body may include an elongate extension configured to be inserted (and/or driven into) the bone.

The clamp may be a manually expandable/contractable clamp (e.g., a ring clamp), or it may be formed of a shape memory material that has a “memorized” smaller diameter allowing it to be clamped onto (or in some cases expanded against) the edge region of the implant. For example, the clamp may comprise a shape memory alloy. In some examples the clamp is configured to apply between 100 and 500 N of retaining force (e.g., between 150-400 N, between 200-400N, about 300 N, etc.).

The one or more sheets of BC material over the engagement surface may be infiltrated with the hydrogel material to form the BC-network hydrogel, so that the BC-network hydrogel is attached to the implant with shear strength that is greater than or equal to native cartilage. The one or more sheets of BC material may be cut to wrap over the edge region so that the one or more sheets of BC material lays flush with a surface of the edge region (e.g., without folds or overlap).

In any of these examples, the engagement surface may have a non-circular perimeter (e.g., oval, rectangular, cartouche, etc.). The engagement surface and/or edge region may be porous.

Also described herein are methods of forming an implant. For example, a method may comprise: placing one or more sheets of a bacterial cellulose (BC) material over an engagement surface of the implant so that a peripheral region of the one or more sheets of BC material fold over an edge region of the implant that is approximately perpendicular to the engagement surface; clamping the peripheral region of the one or more sheets of BC material against the edge region; and infiltrating the one or more sheets of BC material over the engagement surface with a hydrogel material to form a BC-network hydrogel over the engagement surface.

Any of these methods may include cutting the one or more sheets of BC material to fit over the engagement surface and over the edge region. For example, cutting the one or more sheets of BC material to fit over the engagement surface and over the edge region without folding. Any of these methods may include adhesively securing the one or more sheets of BC material to the engagement surface and/or to the edge region with an adhesive. The methods described herein may include curing the adhesive under pressure (e.g., curing the adhesive under between 100 MPa and 500 MPa of pressure). Infiltrating the one or more sheets of BC material over the engagement surface may include infiltrating with polyvinyl alcohol (PVA). In some examples, infiltrating may include infiltrating with PVA and poly(2-acrylamido-2-methyl-1-propanesulfonic acid (PAMPS). Placing the one or more sheets of the BC material over the engagement surface may comprise placing three or more sheets of BC material over the engagement surface.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1A shows two titanium plugs bonded with RelyX Ultimate cement before (top) and after (bottom) shear testing.

FIG. 1B shows two titanium plugs bonded to BC-PVA-PAMPS hydrogel with RelyX Ultimate cement before (top) and after (bottom) shear testing.

FIG. 1C is a graph illustrating the adhesive shear strengths of two titanium plugs bonded with various cements.

FIG. 1D is a graph of the adhesive shear strengths of two titanium plugs bonded to the BC-PVA-PAMPS hydrogel with various cements.

FIG. 1E is a SEM image of the fracture surface in FIG. 1A.

FIG. 1F is a SEM image of the fracture surface in FIG. 1B.

FIG. 2 shows an image of shear test fixture 1, used for shear testing of the plug-to-plug samples shown in FIGS. 1A and B.

FIG. 3 is an SEM image of a cross-section of a bacterial cellulose sheet.

FIG. 4 is an image illustrating one example of a method of attaching a hydrogel to a metallic plug, including using a clamp (e.g., a shape memory alloy clamp) as described herein.

FIG. 5 is an image showing one example of a sheet of bacterial cellulose (BC) cut (e.g., with legs or crenellations) for wrapping over the edge of the metal rod as described herein.

FIGS. 6A and 6B illustrate examples of a fixture that may be used for aligning forming the materials described herein (e.g., aligning the BC, including a rod, cut BC, and ring clamp) as described herein. FIG. 6A shows a perspective view of the fixture. FIG. 6B shows a sectional view through the fixture.

FIG. 7 is an example of one test fixture that may be used to test searing of cartilage off of bone and/or a hydrogel material off of a test rod, as described herein.

FIG. 8A is a graph illustrating the results for shear testing of the samples shown in FIGS. 9A-9D and 10A-10 (n=3 for all measurements). As shown in FIG. 8B, the difference in the 2 layer and 5 layer BC measurements were statistically significant with a p-value<0.05.

FIGS. 9A-9D show images of samples of materials tested (e.g., tested as shown in FIG. 8A). FIG. 9A shows a sample of pig cartilage. FIG. 9B shows an example of a sample of hydrogel without any cement to secure it to the metal substrate. FIG. 9C shows an example of a one-layer cement as described herein. FIG. 9D shows example of a sample in which a clamp was not used to align the BC.

FIGS. 10A-10C show examples of samples test as shown in FIG. 6B, after failure (after the test).

FIG. 11 illustrates one example of a method for attaching a hydrogel (e.g., a BC-PVA hydrogel or in some examples a BC-PVA-PAMPS hydrogel) to an implant (e.g., a titanium implant) for treatment of osteochondral defects.

FIG. 12 illustrates one example of a clamp (shown as a shape memory alloy clamp in a cartouche shape) as described herein.

FIG. 13 schematically illustrates one method of securing a hydrogel to an implant to have a high shear strength, as described herein.

### DETAILED DESCRIPTION

The methods and apparatuses (e.g., devices, systems, etc. including in particular implants) described herein may be used to form an attachment between a hydrogel and a substrate, including (but not limited to) a hydrogel and a metal substrate for part of a medical implant. These methods and apparatuses may include a hydrogel that includes, as part of the hydrogel, a bacterial cellulose. For example, the hydrogel may be a triple network hydrogel that includes a bacterial cellulose material. In general, the bacterial cellulose within the hydrogel may be oriented as described herein so that the bacterial cellulose fibers are generally oriented perpendicular to the substrate to which they are applied. The substrate may be a porous substrate, such as a porous metal (e.g., titanium).

In some examples the apparatuses described herein may form part of a surgical implant for treating a defect, such as an osteochondral defect. For example, a surgical implant may include a surface that is covered in a hydrogel; this surface may act an interface between one or more other body regions, including hard tissues, such as bone and cartilage. Repair of a cartilage lesion with a hydrogel may benefit from long-term fixation of the hydrogel in the defect site. Attachment of a hydrogel to a base (substrate) that allows for integration with bone could enable long-term fixation of the hydrogel, but current methods of forming bonds to hydrogels have less than a tenth of the shear strength of the osteochondral junction. The apparatuses and methods described herein may include bonding a hydrogel to a surface (e.g., base) with a shear strength that is many times larger than has been previously achieved.

Articular cartilage lesions, which most often occur in the knee, typically have a limited intrinsic ability to heal, and are associated with joint pain and disability. The shear strength of an attachment between human cartilage and bone has been reported to be about 7.25±1.35 MPa when tested at the osteochondral junction, or about 2.45±0.85 Mpa when tested at the level of the subchondral bone (both were tested at a shear rate of 0.5 mm/min). This difference in shear strength may explain why subchondral bone fractures are more common than removal of cartilage from bone. Others have measured the shear strength of the mature bovine osteochondral junction to be 2.6±0.58 MPa, albeit at a higher shear rate (38 mm/min). In comparison, cyanoacrylate (“Super Glue”) bonds cartilage with a shear strength of 0.7 MPa. The high shear strength of the osteochondral junction may be attributed to the way in which the collagen nanofibers, which give cartilage its high tensile strength, are mineralized and anchored to the surface of bone with hydroxyapatite.

One way to increase the shear strength (e.g., to 2.28±0.27 MPa) for a hydrogel on titanium may be achieved by first bonding freeze-dried bacterial cellulose (BC, which consists of a network of celluose nanofibers) to titanium with an α-tricalcium phosphate (α-TCP) cement, followed by infiltration of polyvinyl alcohol (PVA), or in some examples PVA and poly(2-acrylamido-2-methyl-1-propanesulfonic acid (PAMPS) into the bacterial cellulose, e.g., to create a BC-PVA-PAMPS hydrogel. This approach, developed as part of the same work described herein, may be referred to as Nanofiber-Enhanced STicking (NEST). Although the shear strength achieved with NEST represented a three-fold increase over the state-of-the-art, described herein are further improvements that may allow the highest values of the shear strengths (e.g., 7.25±1.35 MPa or more) similar to those reported for the human osteochondral junction. In addition, the methods and apparatuses described herein may be directly compared with the shear strength of cartilage with the same test fixture.

The methods and apparatuses described herein may increase the adhesive shear strength between a hydrogel and a substrate (e.g., a metal substrate) so that it matches the shear strength of attachment between cartilage and bone in the same test fixture. To show this, several alternative cements were compared to α-TCP. Although these alternative cements increased the shear strength of attachment between porous titanium plugs, they did not increase the adhesive shear strength between the BC-PVA-PAMPS hydrogel and porous titanium. The work described herein, for the first time, proposed and examines the hypothesis that the shear strength of the hydrogel on titanium is limited by delamination of the cellulose nanofiber layers, which are typically oriented parallel to the direction of the applied shear force. This hypothesis was tested by orienting the cellulose nanofibers perpendicular to the substrate (and therefore oriented perpendicular to the direction of the applied shear force), e.g., by wrapping the BC layer over the sides of the cylindrical metal plug. The bacterial cellulose (BC) layers and the resulting hydrogel were secured in place, e.g., with a shape memory alloy clamp. This change in nanofiber orientation increased the shear strength of attachment to be equivalent to the porcine osteochondral junction in the same test fixture. In this orientation, the shear strength of attachment increased with the number of BC layers, which increased the force required to fracture the hydrogel at the periphery of the plug. This new method of hydrogel attachment will allow the creation of orthopedic devices with surfaces that mimic the properties of cartilage.

### EXAMPLES

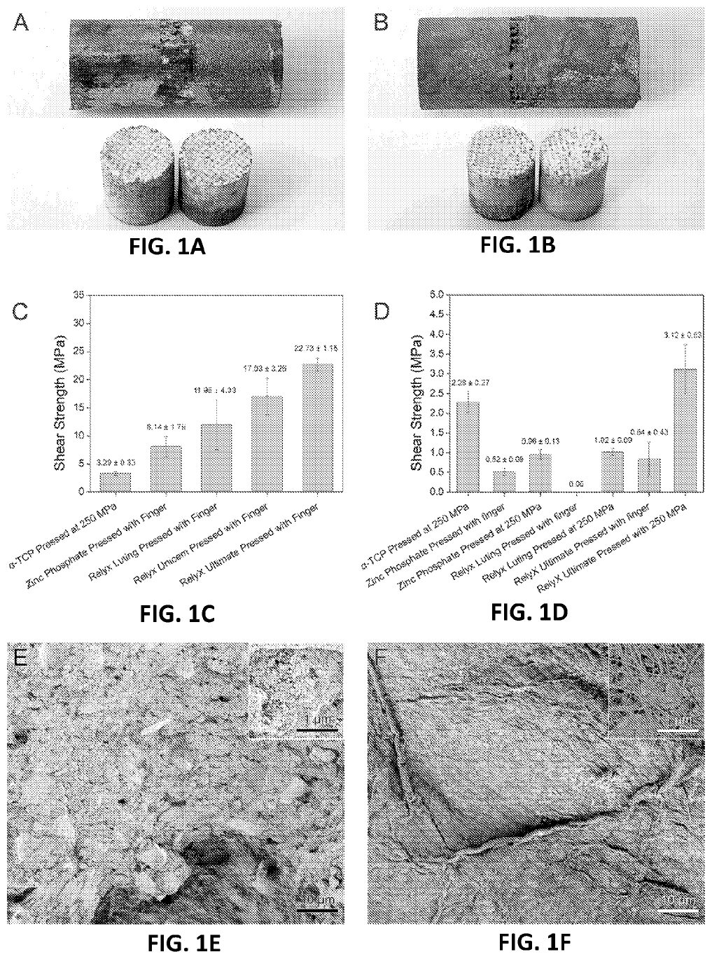

The shear strength of different cements were first tested between two titanium plugs topped with a 1-mm-thick layer of 3D printed struts with a porosity of 70%. In the case of the α-TCP cement, the sandwich structure was pressed together in a die at 250 MPa to reduce the porosity of the cement. The other plugs were pressed together by hand. An example image of a sample made with a RelyX Ultimate cement before and after shear testing is shown in FIG. 1A.

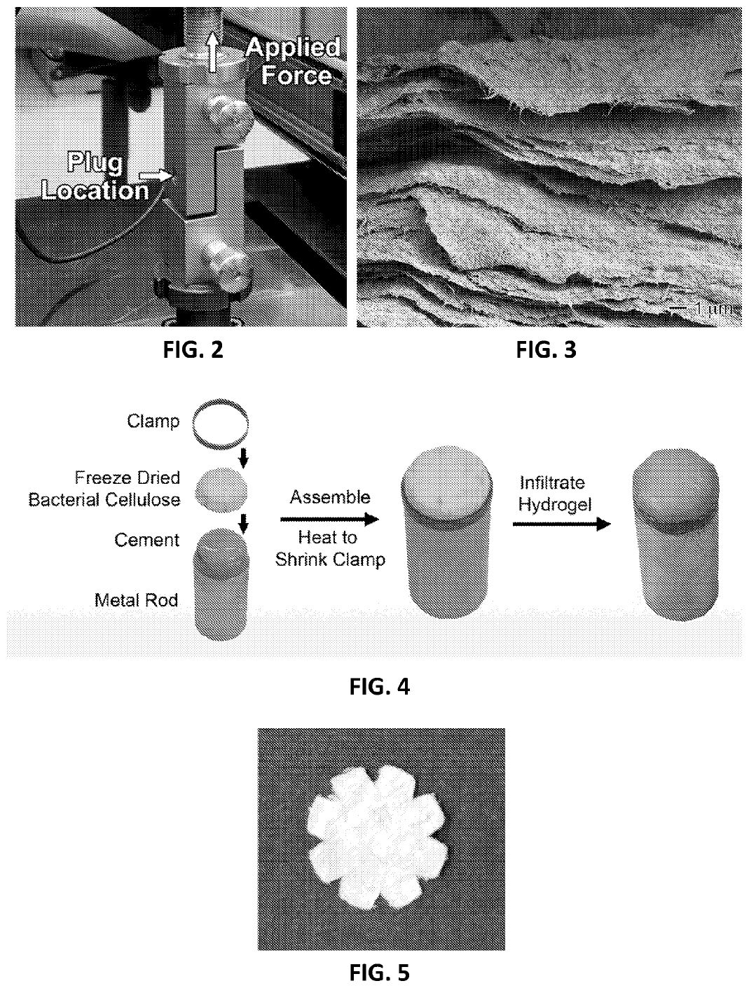

Shear testing was performed on a Test Resources 830LE63 Axial Torsion Test Machine equipped with a 100 lb. load cell and a custom-made shear testing fixture (see FIG. 2). Note that in this “plug-to-plug” sample configuration, the shear force is applied evenly over the entire area of attachment. As shown in FIG. 1B, all the alternative cements that were tested exhibited higher shear strengths than α-TCP cement for bonding two porous titanium plugs. All sample fracture surfaces indicated cohesive failure similar to the RelyX™ Ultimate cement sample shown in FIG. 1A.

The attachment of the same cements to a BC-PVA (or BC-PVA-PAMPS) hydrogel in a sandwich structure was also tested. The sample was prepared by cementing a BC sheet between the titanium plugs. The sample was either pressed together by hand for 2 minutes, or for 1 hour at 250 MPa. In some examples PVA and PAMPS were then infiltrated into the BC to create a hydrogel. An image of a sample prepared with the RelyX™ Ultimate cement is shown in FIG. 1B.

In contrast to the results for bonding porous titanium, none of the alternative cements pressed by hand increased the shear strength of hydrogel attachment relative to α-TCP pressed at 250 MPa. The relatively low strength of attachment may be due to a lack of penetration of the cements into the nanofibrous BC matrix. This hypothesis was tested by pressing the other cements at 250 MPa in the wet state prior to curing, similar to the case of α-TCP. The application of pressure increased the shear strength for each alternative cement. However, none of the shear strengths were significantly greater than that achieved with α-TCP. It was surprising that the RelyX™ Ultimate cement, for example, exhibited a shear strength 6.9 times higher than α-TCP for bonding porous titanium, but did not significantly increase the shear strength for bonding the hydrogel.

Scanning electron microscopy (SEM) images of the fracture surfaces for the porous titanium (FIG. 1E) and hydrogel (FIG. 1F) samples were taken to determine the reason for the lower shear strength of the hydrogel samples. For the porous titanium sample (FIG. 1E), a number of smooth fracture surface are visible in the SEM image, indicating failure was due to the fracture of the cement. However, in the case of the hydrogel sample, no smooth fracture surfaces are visible for the cement. Instead, the SEM image shows the nanofibrous surface of the BC. No fiber pull-out or fiber fracture is readily apparent in the image. Rather, it appears as though failure was due to delamination of the layers of nanofibers in the BC.

In general, the sheer strength of the connection between the hydrogel and the substrate may be dramatically enhanced by including a hydrogel including a bacterial cellulose in which the fibers of the BC have been aligned so that the fibers are perpendicular to the substrate surface.

FIG. 3 shows the layered structure of the BC, e.g., in a sheet of commercially available BC, which is readily apparent when imaged by SEM in a direction perpendicular to the sheet. This sample was prepared by freeze-drying and cutting the BC. The layered nature of BC has been noted in a number of previous studies. It is typically due to the layer-by-layer construction of the BC film by bacteria at the air-liquid interface. Studies of collagen in cartilage have indicated it also has a layered structure, albeit one in which the layers start by being oriented perpendicular to bone and then curve over to be parallel to the cartilage surface. The structure of collagen layers in cartilage suggests the attachment strength of a hydrogel such as a BC-PVA and/or BC-PVA-PAMPS hydrogel can be increased if the BC layers are curved over such that the nanofiber sheets in the BC are oriented perpendicular to the direction of shear. A perpendicular orientation of the nanofiber sheets relative to the direction of shear should increase the shear strength because removal of the hydrogel (e.g., BC-PVA, BC-PVA-PAMPS, etc.) from the titanium may require fracture of the BC nanofibers. In contrast, shear-induced fracture of the hydrogel in a direction parallel to the surface of the BC sheets may involve delamination of the layers and breaking relatively few nanofibers (see, e.g. FIG. 1F). Thus, the connection between the hydrogel and the substrate may be significantly stronger in the plane of the fiber sheets than out of plane. Indeed, the highest shear strength achieved in FIG. 1D (3.12 MPa) is approximately 6 times lower than the tensile strength of the hydrogel, a test which involves nanofiber fracture.

In some examples, the methods for orienting the BC nanofibers in the hydrogel perpendicular to the direction of shear described herein may include wrapping the hydrogel around the periphery of the metal plug and securing the hydrogel in place with a clamp. The clamp may be a shape memory alloy clamp, e.g., initially in a deformed state; upon heating, the clamp may shrink to a memory shape. The clamp may apply a high clamping force. For example, a ring clamp may have a diameter of between 5 mm and 50 mm (e.g., between 10-40 mm, between 15 and 35 mm, etc.), and a ring thickness of between about 0.1 mm and 0.4 mm (e.g., about 0.27 mm), and a height of between about 0.5 mm and 4 mm (e.g., about 1 mm). In one example, the shape memory alloy clamp may, upon heating, provide a nominal clamping force of about 300 N (67 lbf). In some examples a NiTiNb shape memory alloy (Alloy H from Intrinsic Devices, Inc.) was chosen for the clamp due to its convenient operating temperature and large temperature range over which the clamping force is maintained. For this alloy, the full clamping force is obtained at 165° C. and is maintained from −65° C. to +300° C. The NiTiNb alloy is also more corrosion resistant than NiTi, which is used currently in implants, suggesting that it is biocompatible. Alternatively, a NiTi alloy may be used.

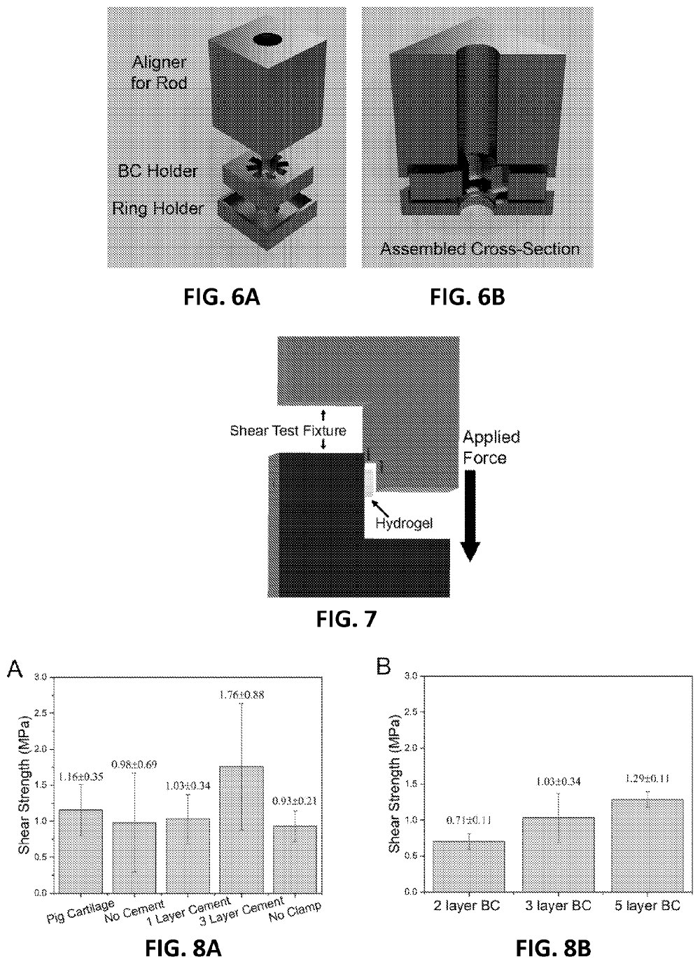

A brief overview of how the hydrogel is attached to a metal base is illustrated in the example of FIG. 4. In this example freeze-dried BC sheets were cut into octagonal shapes with 8 projections (e.g., “legs”) that can be bent over the edges of the implant, as shown in the example of FIG. 5. This cut may remove excess BC that would otherwise be folded up on the sides of the cylinder. The pieces of cut BC were then placed into a fixture, similar to that shown in FIG. 6, that facilitated centering and alignment of the ring clamp with the pieces of BC and the metal rod, which in this case was stainless steel. The metal rod was pushed down through the fixture so that the ring pushed the pieces of BC onto the metal rod. This process of pushing the ring over the BC and onto the rod could also in theory be done by hand. The use of an alignment features, such as shown in FIGS. 6A-6B may help consistently center the pieces during assembly. The sample may then be clamped, e.g., by heated in an oven at 90° C. to initiate clamping in a shape-memory alloy material preset as described herein (which starts at a temperature of 50° C.). The part was then heated in a hydrothermal bomb at 120° C. for 24 hours with PVA to infiltrate the polymer into the BC. Finally, in some examples the BC-PVA was infiltrated with PAMPS by soaking in a solution of 30% AMPS (2-acrylamido-2-methylpropanesulfonic acid) with 9 mg/mL MBAA crosslinker, 5 mg/mL 12959 and 0.5 mg/mL KPS for 24 hours. The sample was cured with UV for 15 minutes, followed by curing at 60° C. for 8 hours for heat curing.

The clamp and/or substrate may be configured to prevent breaking the bacterial cellulose. For example, the distance between the inner diameter of the ring and the outer diameter of the rod may be adjusted to achieve a high clamping force without breaking the BC. For attachment of three pieces of BC to the metal rod in one example, the outer diameter of the rod was about 5.7 mm, and the inner diameter of the ring was about 6.4 mm, leaving 0.7 mm for the three pieces of BC. Each piece of frieze-dried BC was 0.136±0.026 mm, leaving 0.3 mm of space. The ring can shrink to a diameter of 6.15 mm to consume this space and firmly clamp the BC onto the metal. In addition, the BC will expand by about 0.2 mm after infiltration of the hydrogel components. Note that the tolerances of the parts are ±0.13 mm. Reducing the space between the rod and the ring in some cases led to a greater failure rate due to breaking the legs off the BC when the ring was pushed across the BC layers. Using a larger distance between the rod and ring led to a less secure attachment of the hydrogel to the rod. Through trial and error, we found that leaving about 0.23 mm of clearance for each piece of BC was sufficient to firmly clamp the BC in place without breaking off the BC legs when sliding the ring over the BC.

The strength of attachment of the hydrogel to the rod was then compared to the strength of attachment of cartilage to bone. This was not possible with the plug-to-plug configuration used for the samples in FIGS. 1A-1B, because there is no way to attach a rod to the surface of cartilage with the same strength as the osteochondral junction. Previous tests of the shear strength of the osteochondral junction have used an L-shaped jig that pulls a square-shaped piece of cartilage off bone while constraining the movement of cartilage in a direction perpendicular to the shear plane. To perform a similar test with the cylindrical specimen described herein, a shear test fixture such as the one shown in FIG. 7 was used. The test specimen was secured in a cylindrical hole in the left side of the fixture. The right side of the fixture was machined to have a complementary half-cylinder that was used to push the hydrogel or cartilage off of their substrates. A crosshead displacement rate of 2 mm/min was used for all the measurements. While fixture 1 in FIG. 2 applies the shear force relatively evenly over a given interface, fixture 2 in FIG. 7 focuses the applied force on one edge of the rod. This difference in the manner in which the force is applied is expected to lead to a lower observed shear force in fixture 2 relative to fixture 1, especially since fixture 2 can potentially cause cleavage and peel streses.

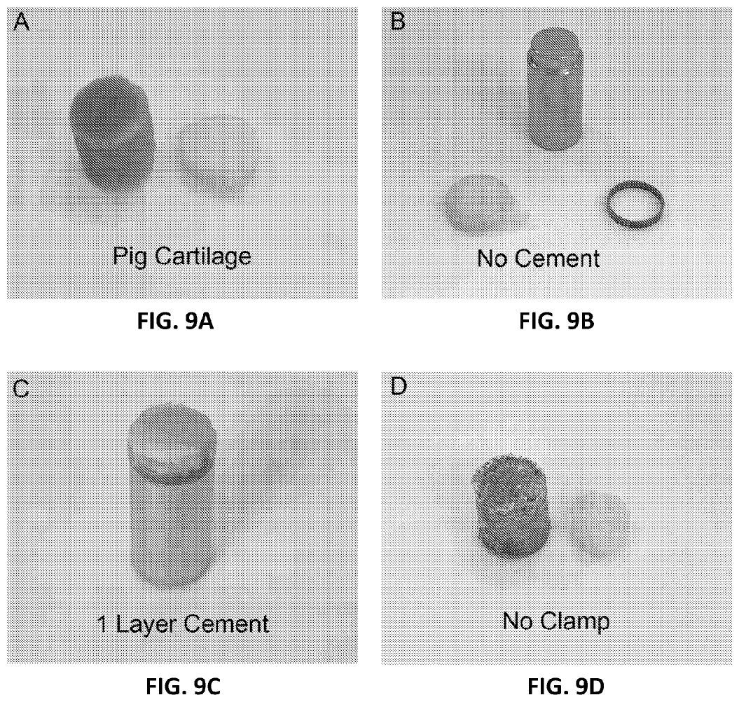

FIGS. 8A-8B show the results for shear testing samples with shear test fixture 2. Pig cartilage had an average shear strength of about 1.16±0.35 MPa. FIG. 9A shows the cartilage was sheared cleanly off of the underlying bone in this sample. The lower shear strength of cartilage measured with fixture 2 relative to previous work (2.45±0.85 to 2.6±0.58 MPa) was likely due to the cylindrical shape of our specimens, which may concentrate stress over a smaller area at the edge of the specimen compared to the rectangular specimens tested previously. Although stress concentration was at least partially avoided by shearing the sample with a matching cylindrical surface, some concentration of shear stress may still be present. In addition, there may have been some peeling and/or cleavage in addition to shear due to imperfect alignment or imperfect constraining of the cartilage from moving out of the shear plane. Although a lower number was obtained for the shear strength of cartilage than previous authors, the direct comparison with hydrogel samples in the same fixture provided herein still provide a valid answer to the question of whether cartilage-equivalent shear strength was achieved. Further, the standard deviation of the cartilage shear strength measurements described herein are lower than those obtained previously, indicating the measurement method is at least as precise as previous efforts.

By using a clamp (e.g., a shape memory material clamp) without cement, a hydrogel sample with three layers of BC was attached to the metal rod with a shear strength of 0.98 MPa. We note that this result is within the error of the average shear strength for pig cartilage, indicating cartilage-equivalent shear strength can be achieved with the clamp alone. FIG. 9B shows the attachment failed due to the hydrogel being pulled out of the clamp. The hydrogel was also dented where it was contacted by the shear fixture (not visible in FIG. 9B).

The addition of 1 layer of cement between the hydrogel and the metal rod further increased the strength of attachment, to 1.03±0.34 MPa. However, the increase in this example cannot be said to be statistically significant given the standard deviation of the measurements. This result indicates the addition of cement underneath the first layer of BC does not significantly increase the strength of attachment beyond what was achieved with the clamp alone. The addition of cement did change the failure mode to fracture of the hydrogel (as shown in FIG. 9C) rather than pull-out of the hydrogel from the clamp (as shown in FIG. 9B). The failure mode in FIG. 9C is preferable in the context of a hydrogel-capped implant because the hydrogel is still covering the metal and not exposing an opposing cartilage surface to wear by a metallic surface, for which the coefficient of friction is higher than the hydrogel.

Next, a sample with 3 layers of cement, one layer beneath each of the three BC layers, was examined. In this case the average strength increased to 1.76±0.88 MPa. This average shear strength exceeds that measured for the pig cartilage samples, but the standard deviation makes the difference in measurements not statistically significant. The addition of cement in between the layers may have increased the average strength by creating a layered composite of cement between each of the BC layers that reinforced the hydrogel. The failure mode for this sample (FIG. 8D) was similar to the case of 1 layer of cement.

A sample in which the hydrogel was attached to the surface of the metal rod with only the cement and not the clamp was also examined. This sample proved impossible to make because, without the clamp, the hydrogel detached from the metal pin during the PVA infiltration process. Presumably the expansion of the hydrogel during PVA infiltration created a sufficient shear force to detach the hydrogel from the surface of the smooth metal rod. Instead, we attached 3 layers of hydrogel to a porous titanium plug with cement between each layer. This sample was prepared with Rely X Ultimate cement and was pressed at 250 MPa, similar to the best result in FIG. 1D. Shear testing of this “no clamp” sample with fixture 2 yielded a shear strength of 0.93±0.21 MPa. Note that this shear strength is 3.4 times lower than the shear strength of 3.12±0.63 measured with fixture 1, indicating that method of measurement used with fixture 2 leads to a lower observed shear strength for a sample with an identical interface. FIG. 9D shows this sample failed cohesively in a similar manner as the cartilage sample. We note that while this shear test result without the clamp was lower than the samples tested with the clamp and cement, the difference was not statistically significant.

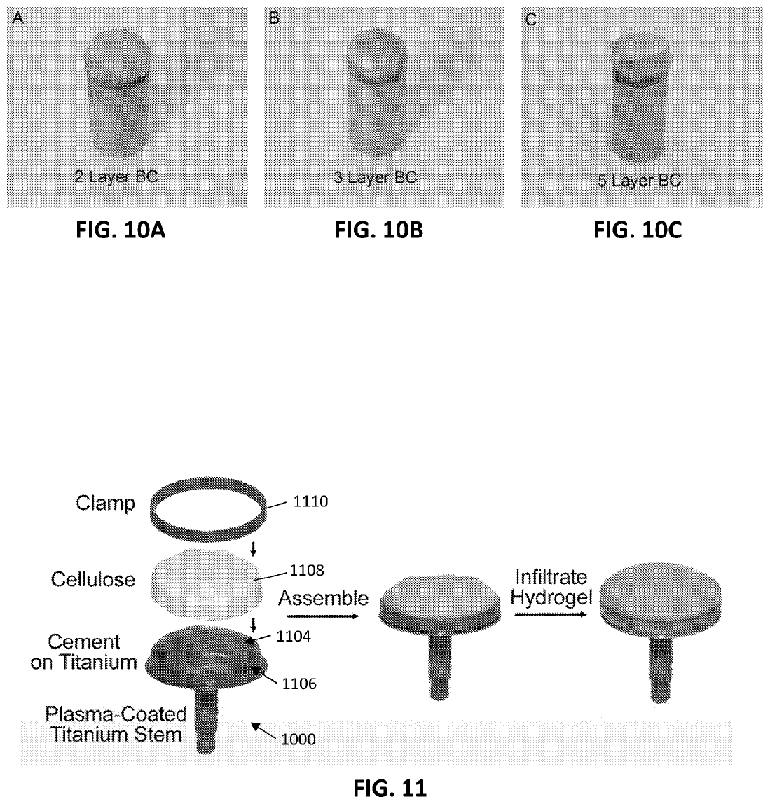

The way in which the samples made with the shape memory alloy clamp fractured (see for example FIG. 9C) suggests that the shear strength of the samples is limited by the tensile force required to fracture the hydrogel that is curved over the edge of the implant. The shear test pushes the hydrogel off of the metal pin, which creates a tensile force on the hydrogel that is clamped around the sides of the pin. This tensile force can either pull the hydrogel out of the clamp (as shown in FIG. 9B) or cause the hydrogel around the periphery of the pin to fracture (as shown in FIG. 9C). These results suggest that the shear strength of attachment can be increased by making the hydrogel layer thicker. FIG. 8B shows the results testing this hypothesis. As expected, the shear strength of attachment increases as the number of BC layers is increased from two to five. Each of these samples had one layer of cement in between the BC layer and the metal pin, and all failed cohesively (see, e.g., FIGS. 10A-10C). The p-value from one-way ANOVA for the 2 layer vs. 5 layer result is 0.039, indicating the difference in these results is statistically significant (p<0.05). The shear strength of the five-layer BC hydrogel was one standard deviation above the average shear strength of the pig cartilage.

Thus, described herein are hydrogel-capped implants. In some examples the hydrogel-capped implants may include a BC containing hydrogel that is clamped to a portion of the implant that is perpendicular to the tissue-engaging surface of the implant. Alternatively or additionally, the BC containing hydrogel may be bonded via an adhesive) to a portion of the implant that is perpendicular to the tissue-engaging surface; the adhesive may be cured under pressure (e.g., under between about 150 MPa and 500 MPa, e.g., about 250 MPa). Any of these apparatuses may include multiple layers of BC that may (optionally) be adhesively secured together.

For example, any of these apparatuses may include a clamp that is used to secure a sheet of BC to the implant; the hydrogel may be formed in the BC so that the BC is part of a network (e.g., triple network hydrogel). Thus, the final apparatus may include a clamp as described herein. In some examples, these clamps may be shape-memory alloy clamps, formed as rings or loops that can be produced in a variety of shapes and sizes for clamping hydrogels to the surface of implants for repair of osteochondral defects. FIG. 11 shows an example of a clamp to attach the hydrogel (e.g., BC-PVA hydrogel, and/or BC-PVA-PAMPS hydrogel) to an osteochondral implant with a diameter of 20.64 mm. In this example five pieces of BC were cut into shapes with 8 octagonal legs that allowed the legs to fold over the edge of the implant. A 0.25-mm-thick coating of commercially pure titanium was applied to the stem of the implant and underneath the base with a plasma spray process in order to improve integration with bone. The top surface of the implant is curved to match the native curvature of the knee. Once the one or more layers of BC are applied and clamped onto the implant, as shown in the middle of FIG. 11, the BC may be infiltrated with the remaining hydrogel components (e.g., PVA, PAMPS, etc.) to form the final hydrogel, as described herein.

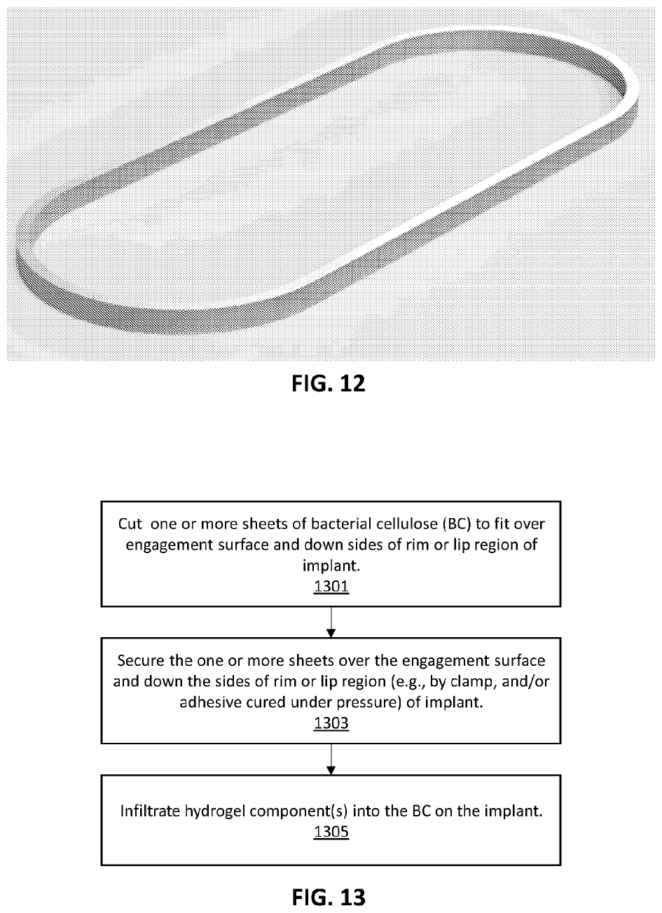

Any appropriate shape or dimensions of the implant may be used. For example, if a patient is suffering from an elongated or oblong cartilage defect that cannot be adequately treated with a circular device, a rectangular, oval, or cartouche-shaped device can be used to treat the defect. FIG. 12 shows an example of a cartouche-shaped clamp that can be used to press an appropriately cut piece of BC onto a cartouche-shaped implant.

In the examples shown and described herein the Bacterial Cellulose (BC) was formed as a sheet. For example, a sheet of BC (Gia Nguyen Co. Ltd.) may be combined and manipulated dry and attached to the substrate as described herein. In some examples (e.g., forming BC-PVA-PAMPS hydrogels), Poly(vinyl alcohol) (PVA) (fully hydrolyzed, molecular weight: 145,000 g mol-1), N,N′-methylenediacrylamide (MBAA, 97.0%), 2-hydroxy-4′-(2-hydroxyethoxy)-2-methylpropiophenone (I2959), potassium persulfate (KPS), 2-acrylamido-2-methylpropanesulfonic acid sodium salt (AMPS, 50 wt. % solution in water) and phosphoserine (e.g., Sigma Aldrich) may be used. Phosphate buffered saline (PBS) may be used for rinsing and hydrating. Examples of adhesives that may be used include, e.g., Ti-6A1-4V ELI (Grade 23) powder (3D Systems), α-tricalcium phosphate (α-TCP) (Goodfellow Corporation), Zinc phosphate cement (Prime-Dent), RelyX Luting 2 (3M ESPE), RelyX Unicem (3M ESPE), RelyX Ultimate cement (3M ESPE) and Scotchbond Adhesive (3M ESPE).

In general, the implant forming the substate may be any appropriate, biocompatible material, include metals and polymers. For example, in some cases titanium may be used. Titanium plugs in FIGS. 4 and 9A-10C were fabricated via selective laser melting (SLM) of Ti-6A1-4V ELI powder on a titanium substrate in an inert argon atmosphere using a 3D Systems DMP ProX 320. Plugs (test samples) were designed to have a diameter of 6 mm and a height of 6.35 mm. The final implants described herein may be any appropriate shape and size. In these examples, the top 1 mm of the plug was composed of a porous strut structure with a porosity of 70%, which may help with adhesive bonding, when adhesives are used (optionally). After printing, the samples were removed from the build plate via wire electrical discharge machining and cleaned by sonication for 15 min in DI water to remove the excess unadhered powder.

As described above, several cements were tested between two porous titanium plugs. To prepare the sample with α-TCP cement, a dry cement mixture consisting of 0.040 g phosphoserine (PPS), 0.312 g of α-TCP and 0.048 g of stainless-steel powder (SSP) was placed into a small dish, 0.140 ml of water was added, and the powder was rapidly mixed with the water. Approximately 0.150 ml of the wet cement mixture was added on top of a porous titanium plug in a metal die with an inner diameter of 6 mm. A second titanium plug was immediately placed into the die with the porous layer in contact with the wet cement, and the sandwich structure was pressed together for 1 hour at 250 MPa. The sample was placed into water at 85° C. for at least 24 hours to facilitate the transformation of α-TCP into hydroxyapatite and was stored in water until just prior to shear testing.

To prepare the sample with zinc phosphate cement, approximately 1 g of the liquid were mixed with 2 g of powder for 90 seconds. The addition of the powder into the liquid was carried out slowly, smoothly and carefully with constant stirring. After that, approximately 0.150 ml of the wet zinc phosphate cement mixture was added on top of the first porous titanium plug in a metal die with an inner diameter of 6 mm. The second titanium plug was immediately placed into the die with the porous layer in contact with the wet cement, and the sandwich structure was pressed together for 1 hour at 250 MPa with a hydraulic press or for 2 minutes by hand. After the cement was completely dry (˜2 hours), the sample was placed into water at 22° C. for at least 24 hours and was stored in water until just prior to shear testing.

To prepare the sample with RelyX™ Luting 2 and RelyX™ Unicem cement, approximately 0.150 ml of the wet RelyX™ Luting 2 or RelyX™ Unicem cement mixture was added on top of the first porous titanium plug in a metal die with an inner diameter of 6 mm. The second titanium plug was immediately placed into the die with the porous layer in contact with the wet cement, and the sandwich structure was pressed together for 1 hour at 250 MPa with a hydraulic press or for 2 minutes by hand. The sample was placed into water at 22° C. for at least 24 hours was stored in water until just prior to shear testing.

To prepare the sample with RelyX™ Ultimate cement, Scotchbond Adhesive was first applied to the porous surfaces of both titanium plugs. The adhesive was allowed to set for 20 s before being blown by air for another 5 s. After that, approximately 0.150 ml of the wet RelyX™ Ultimate cement mixture was added on top of the first porous titanium plug in a metal die with an inner diameter of 6 mm. The second titanium plug was immediately placed into the die with the porous layer in contact with the wet cement, and the sandwich structure was pressed together for 1 hour at 250 MPa with a hydraulic press or for 2 minutes by hand. The sample was placed into water at 22° C. for at least 24 hours and was stored in water until just prior to shear testing.

As mentioned, all of the hydrogel samples described herein were made with freeze-dried BC. BC sheets were cut and placed between 2 metal plates. A 6.59 kg weight was applied to the metal plate to flatten the BC sheets. The BC sheets were frozen at −80° C. and then in liquid nitrogen. Note that if the BC sheets are placed directly into liquid nitrogen without the pre-freezing step they fractured. The BC sheets were then removed and freeze-dried at −78° C. for 24 h.

Samples for Hydrogel Plug-to-Plug Shear Testing were made using each of several cements to attach the hydrogel between two porous titanium plugs to test the adhesive shear strength. For the α-TCP sample, a cement mixture consisting of 0.080 g PPS, 0.624 g of α-TCP, and 0.096 g of SSP was placed into a small dish, 0.280 ml of water was added, and the powder was rapidly mixed with the water. Then 0.150 ml of the wet cement mixture was added on top of the porous titanium plug in the die. The Freeze-Dried BC sheet was then placed on top of the cement in the die, and an additional 0.150 ml of the wet cement mixture was added on top of the BC sheet. A second porous titanium plug was then placed on top of the Freeze-Dried BC sheet in the die to create a sandwich structure. The sandwich structure was pressed for 1 hour at 250 MPa. The sample was placed into water at 85° C. for 24 hours to facilitate the transformation of α-TCP into hydroxyapatite. The sample was then placed into a hydrothermal reactor with a mixture of PVA (40 wt. %) and DI water (60 wt. %) to infiltrate PVA into the BC layer. The sample was frozen at −78° C. and thawed to room temperature to further increase the strength of the hydrogel. The sample was then soaked in a solution containing AMPS, (30 wt. %) cross-linker (MBAA, 60 mM), and heat initiator (potassium persulfate, 0.5 mg ml⁻¹) for 24 hours. The hydrogel was heat cured at 60° C. for 8 hours and the sample was soaked in DI water for at least 24 hours.

For the zinc phosphate cement, approximately 1 g of the liquid were being mixed with 2 g of powder for 90 seconds. The addition of the powder into the liquid was carried out slowly, smoothly and carefully with constant stirring. Approximately 0.150 mL of the wet zinc phosphate cement mixture was added on top of the first porous titanium plug in a metal die with an inner diameter of 6 mm. The BC sheet was then placed on top of the cement in the die, and an additional 0.150 mL of the wet cement mixture was added on top of the BC sheet. The second porous titanium plug was then placed on top of the BC sheet in the die to create a sandwich structure. The sandwich structure was pressed for 1 hour at 250 MPa or for 2 minutes by hand. After the cement was completely dry (˜2 hours), the sample was placed into water at 22° C. for 24 hours to rehydrate the BC. The sample was then placed into a hydrothermal reactor with a mixture of PVA (40 wt. %) and DI water (60 wt. %) to infiltrate PVA into the BC layer. The sample was frozen at −78° C. and thawed to room temperature to further increase the strength of the hydrogel. The sample was then soaked in a solution containing AMPS, (30 wt. %) cross-linker (MBAA, 60 mM), and heat initiator (potassium persulfate, 0.5 mg ml⁻¹) for 24 hours. The hydrogel was heat cured at 60° C. for 8 hours and the sample was soaked in DI water for at least 24 hours.

For the RelyX™ Luting 2 and RelyX™ Unicem cement, approximately 0.150 mL of the wet RelyX™ Luting 2 or RelyX™ Unicem cement mixture was added on top of the first porous titanium plug in a metal die with an inner diameter of 6 mm. The BC sheet was then placed on top of the cement in the die, and an additional 0.150 mL of the wet cement mixture was added on top of the BC sheet. The second porous titanium plug was then placed on top of the BC sheet in the die to create a sandwich structure. The sandwich structure was pressed for 1 hour at 250 MPa or for 2 minutes by hand. The sample was placed into water at 22° C. for 24 hours to rehydrate the BC. The sample was then placed into a hydrothermal reactor with a mixture of PVA (40 wt. %) and DI water (60 wt. %) to infiltrate PVA into the BC layer. The sample was frozen at −78° C. and thawed to room temperature to further increase the strength of the hydrogel. The sample was then soaked in a solution containing AMPS, (30 wt. %) cross-linker (MBAA, 60 mM), and heat initiator (potassium persulfate, 0.5 mg ml⁻¹) for 24 hours. The hydrogel was heat cured at 60° C. for 8 hours and the sample was soaked in DI water for at least 24 hours.

For the Relyx™ Ultimate cement, Scotchbond Adhesive was first applied to the porous surfaces of both titanium plugs and both surfaces of a BC sheet. The adhesive was allowed to set for 20 seconds before being blown by air for another 5 seconds. After that, approximately 0.150 mL of the wet RelyX™ Ultimate cement mixture was added on top of the first porous titanium plug in a metal die with an inner diameter of 6 mm. The BC sheet was then placed on top of the cement in the die, and an additional 0.150 mL of the wet cement mixture was added on top of the BC sheet. The second porous titanium plug was then placed on top of the BC sheet in the die to create a sandwich structure. The sandwich structure was pressed for 1 hour at 250 MPa or for 2 minutes by hand. The sample was placed into water at 22° C. the rehydrate the BC. The sample was then placed into a hydrothermal reactor with a mixture of PVA (40 wt. %) and DI water (60 wt. %) to infiltrate PVA into the BC layer. The sample was frozen at −78° C. and thawed to room temperature to further increase the strength of the hydrogel. The sample was then soaked in a solution containing AMPS, (30 wt. %) cross-linker (MBAA, 60 mM), and heat initiator (potassium persulfate, 0.5 mg ml⁻¹) for 24 hours. The hydrogel was heat cured at 60° C. for 8 hours and the sample was soaked in DI water for at least 24 hours.

For preparing the pig cartilage samples used for the shear test, the pig knee was first clamped on a bench vise. An osteochondral autograft transfer system (OATS) tool was used harvest the osteochondral plug from the pig knee. The OATS donor harvester was positioned on the pig knee surface and tamped approximately 15 mm into the surface. The handle was rotated to harvest the plug and withdrawn. The pig plug was extruded out by the core extruder. The pig plug was cut to make the bone region 8 mm bone in length.

Preparation of all hydrogel samples started with cutting the freeze-dried BC. The freeze-dried BC was placed on a cutting mat that had been made to be sticky with PVA glue. The BC was cut in the shape of an octagon with an inner diameter of D mm and 8 legs which has leg lengths of L mm and widths of W=0.383 D. The sample was labeled as BC-D-L after cutting. The 8-piece star shape (BC-D-L) was generated by MATLAB and loaded into Silhouette Studio software. For example, the 3 layers BC shear test sample was fabricated with BC-6-2, BC-6-1.75, and BC-6-1.75 from top to bottom. The following cutting settings were used in Silhouette Studio: Force=3, Speed=1, and Passes=3. After cutting, the BC was removed and placed in petri dish.

For adhering three pieces BC to the shear test rod with one layer of cement and a clamp, a stainless-steel test rod was machined to have a top section with a diameter of 5.7 mm and a height of 2 mm, and a bottom section with a diameter of 17 mm and a height of 13 mm. The three pieces of cut BC were placed in an alignment fixture. Scotchbond Universal Adhesive was applied to the layer of the BC in contact with the rod and the top surface of the rod. The adhesive was allowed to set for 20 seconds before being blown by air for another 5 seconds. About 0.15 g RelyX Ultimate Cement was then applied to same surfaces coated with the Scotchbond Universal Adhesive. The rod was pressed into the BC layers and then into the ring clamp. The cement was cured for 1 h. The samples were heated in an oven at 90° C. for 10 min to shrink the clamp. The sample was then soaked in DI water for 1 hr. in a centrifuge tube.

For creating the sample without the cement, the same procedure was followed as above but no adhesive or cement was applied to the BC or the rod. For creating samples with three layers of cement, additional adhesive and cement was applied as described above to between each layer of BC, in addition to between the BC and the rod. For testing samples with 2 layers of BC, the top diameter of the rod was 5.8 mm instead of 5.7 mm, and the size of the cut BC layers were BC-6-1.75 and BC-6-1.75. For testing samples with 5 layers of BC, the top diameter of the rod was 5.2 mm, and the size of the cut BC layers were BC-3-2, BC-3-2, BC-5.5-2.25, BC-5.5-2, and BC-5.5-1.75.

After attachment of BC to the metal rod, all hydrogel samples were made by infiltrating, e.g., PVA and PAMPS into the BC. For infiltration of PVA, the rehydrated sample was placed in a hydrothermal bomb with 40% PVA and 60% DI water. The hydrothermal bomb was heated at 120° C. for 24 h to infiltrate PVA into the BC layers. After 24 h, the hydrothermal bomb was removed from the oven and opened while it was hot. The sample was taken out from the bomb and the extra PVA around the sample was manually removed. The sample was placed into a −80° C. freezer and taken out from the freezer after 30 minutes. The sample was thawed to room temperature before the next step, infiltration of PAMPS. The thawed sample was put into a 30% AMPS (2-acrylamido-2-methylpropanesulfonic acid) solution with 9 mg/mL MBAA crosslinker, 5 mg/mL 12959 and 0.5 mg/mL KPS for 24 h (all fully dissolved). The sample was taken out and cured with UV for 15 minutes. It was transferred to an air-tight centrifuge tube and placed into a 60° C. oven for 8 h for heat curing. After curing, the implant was placed in PBS for rehydration.

Shear testing, such as that shown in FIGS. 8A-8B, was performed on a 830LE63 Axial Torsion Test Machine equipped with a 100 lb. load cell. Each test was performed in customized shear test fixtures. For shearing of cartilage or hydrogel on metal samples, the sample was secured in a cylindrical hole in the left side of the fixture. The hole size was 6 mm for the pig plug and 7 mm for the hydrogel samples. Spacers were added underneath the samples to precisely align the shear plane to the cartilage-bone or hydrogel-metal interface. The right side of the fixture was machined to have a complementary half-cylinder that was used to push the hydrogel or cartilage off of their substrates. The diameter of the right half-cylinder matched that of the left side (either 6 or 7 mm). Rubber was placed between the sample and the right shear fixed to apply some pressure during the shear test in order to minimize cleavage and peeling. A crosshead displacement rate of 2 mm min-1 was used for all the measurements.

The methods and apparatuses described herein include a method of forming an implant including a hydrogel on an engagement surface of the implant. The engagement surface may be configured to engage a hard tissue (e.g., bone) or another implant, once inserted into a body. Although examples of implants include a bone implant 1100 such as the one shown in FIG. 11, any implant configuration may be used. In general, the implant includes an engagement surface. The engagement surface may be convex, concave, flat, or otherwise curved or shaped. The engagement surface typically includes a lip or rim region that extends approximately perpendicularly (e.g., between 70 degrees and 140 degrees, e.g., between 70 degrees and 110 degrees, approximately 90 degrees, etc.) relative to the engagement surface. The in the example shown in FIG. 11, the engagement surface is shown with a cement applied on the surface 1104, and an edge (also referred to as a rim or lip region) 1106 surrounds the engagement surface. As discussed above, one or more sheets of BC 1108 may be cut to fit over the engagement surface and down (or in the case of recessed engagement surfaces, up) the side of the approximately perpendicular edge region. In FIG. 11, the clamp 1110 may fit over the BC and edge and be activated to clamp down onto the one or more layers of BC to secure them against the edge, as shown.

FIG. 13 schematically illustrates one example of a method of securing a hydrogel (e.g., a BC containing hydrogel) onto an implant, as described herein. This method may provide a process that enables the attachment of a hydrogel to the surface of an implant (e.g., an orthopedic implant) with approximately the same or greater shear strength as the natural cartilage-bone interface. In some examples, clamping the hydrogel around the periphery of the engagement surface of the implant reorients the nanofibers in the BC so that they are perpendicular to the direction of shear. This reorientation increases the average strength of attachment by necessitating fracture of the nanofiber sheets to shear the hydrogel off the implant. Without this reorientation, the BC layers may delaminate, resulting in a lower shear strength. The methods described herein, may include clamping in conjunction with adhesive cements to further improve the strength of attachment and prevent the hydrogel from being pulled out of the clamp. The shear strength also increased with the number of BC layers used in the hydrogel, indicating the shear strength is limited by the tensile force required to fracture the hydrogel at the periphery of the implant.

For example, in FIG. 13, the one or more sheets of BC (e.g., freeze-dried BC) may be prepared before attaching to the implant. In some examples, the one or more sheets of BC may be cut so that it/they may fit over the engagement surface and may fold over the edge (e.g., lip or rim region) so that pressure may be applied uniformly against the portion of the sheet that is extending over (or in some cases around) the edge region 1301.

The edge region (lip or rim) may be any appropriate size, such as between 0.1 mm and 4 mm (e.g., between 0.2 mm and 3 mm, between 0.4 mm and 3 mm, etc.). The portion of the sheet of BC that extend over the edge region (e.g., lip or rim) may be cut, notched or otherwise formed to prevent substantial folding which may result in uneven pressure and securing force, e.g., by a clamp.

In any of these examples an adhesive, such as one or more of the adhesives described herein, may be applied to the implant before applying the sheet(s) of BC. For example, adhesive may be applied to the engagement surface and/or to the edge or rim region.

The one or more sheets may then be secured over the engagement surface and against the surrounding side(s) (e.g., the lip or rim region) by a clamp and/or by an adhesive 1303. If an adhesive is used it may be cured under pressure for an appropriate time period (e.g., under between about 100-500 MPa for greater than 4 hours, etc.). In some examples the clamp may be a ring or annulus (e.g., collet) of a shape memory alloy material that is configured to transition from a wider configuration to a shape-set narrower configuration once applied over the edge region. In variations in which the engagement surface is recessed and the edge region is raised above the engagement surface, the clamp may be expanded from a narrow to a larger, expanded diameter. The clamp may be configured to apply an amount of force sufficient to retain the sheet(s) in position, but not so large that they cut or damage the BC material.

Thereafter, the hydrogel, including the BC material of the one or more sheets of BC may be infiltrated with the remaining hydrogel component(s) to form the complete hydrogel 1305, such as a triple-network hydrogel, including the BC.

The methods of hydrogel attachment described herein can be used to create hydrogel-coated orthopedic implants with surfaces that mimic the mechanical and tribological properties of cartilage, and bases that enable integration with bone for long-term fixation.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. An implant, comprising:
an implant body, the implant body having an engagement surface surrounded by an edge region that is substantially perpendicular to a perimeter of the engagement surface;
one or more sheets of bacterial cellulose (BC) applied over the engagement surface and along the edge region; and
a clamp securing the one or more sheets of BC between the edge region and the clamp,
wherein the one or more sheets of BC over the engagement surface are infiltrated with a hydrogel material to form a BC-network hydrogel.

2. The implant of claim 1, wherein the implant body is titanium.

3. The implant of claim 1, wherein the engagement surface is curved.

4. The implant of claim 1, further comprising an adhesive on one or more of the engagement surface and the edge region.

5. The implant of claim 1, wherein the one or more sheets of bacterial cellulose comprise 3 or more sheets.

6. The implant of claim 1, wherein the hydrogel material comprises polyvinyl alcohol (PVA).

7. The implant of claim 1, wherein the hydrogel material comprises polyvinyl alcohol (PVA) and poly(2-acrylamido-2-methyl-1-propanesulfonic acid (PAMPS).

8. The implant of claim 1, wherein the implant body comprises an elongated nail shape configured to be implanted into bone.

9. The implant of claim 1, wherein the clamp comprises a shape memory alloy.

10. The implant of claim 1, wherein the clamp is configured to apply between 100 and 500 N of retaining force.

11. The implant of claim 1, wherein the one or more sheets of BC over the engagement surface are infiltrated with the hydrogel material to form the BC-network hydrogel, so that the BC-network hydrogel is attached to the implant with shear strength that is greater than or equal to native cartilage.

12. The implant of claim 1, wherein the one or more sheets of BC is cut to wrap over the edge region so that the one or more sheets of BC lays flush with a surface of the edge region.

13. The implant of claim 1, wherein the engagement surface has a non-circular perimeter.

14. The implant of claim 1, wherein one or more of the engagement surface and edge region are porous.

15. A method of forming an implant, the method comprising:
placing one or more sheets of a bacterial cellulose (BC) material over an engagement surface of the implant so that a peripheral region of the one or more sheets of BC material fold over an edge region of the implant that is approximately perpendicular to the engagement surface;
clamping the peripheral region of the one or more sheets of BC material against the edge region; and
infiltrating the one or more sheets of BC material over the engagement surface with a hydrogel material to form a BC-network hydrogel over the engagement surface.

16. The method of claim 15, further comprising cutting the one or more sheets of BC material to fit over the engagement surface and over the edge region.

17. The method of claim 15, further comprising cutting the one or more sheets of BC material to fit over the engagement surface and over the edge region without folding.

18. The method of claim 15, further comprising adhesively securing the one or more sheets of BC material to one or more of the engagement surface and to the edge region with an adhesive.

19. The method of claim 18, further comprising curing the adhesive under pressure.

20. The method of claim 18, further comprising curing the adhesive under between 100 MPa and 500 MPa of pressure.

21. The method of claim 15, wherein infiltrating the one or more sheets of BC material over the engagement surface comprises infiltrating with polyvinyl alcohol (PVA).

22. The method of claim 15, wherein infiltrating the one or more sheets of BC material over the engagement surface comprises infiltrating with polyvinyl alcohol (PVA) and poly(2-acrylamido-2-methyl-1-propanesulfonic acid (PAMPS).

23. The method of claim 15, wherein placing the one or more sheets of the BC material over the engagement surface comprises placing three or more sheets of BC material over the engagement surface.
